(19) United States
(12) Patent Application Publication
(10) Pub. No.: US 2024/0385188 A1
(43) Pub. Date: Nov. 21, 2024

(54) LABELING AND ANTI-DRUG ANTIBODY ASSAYS FOR AAV VECTORS

(71) Applicant: Regeneron Pharmaceuticals, Inc., Tarrytown, NY (US)

(72) Inventors: Byung Chul Kim, River Vale, NJ (US); Kun Lu, Scarsdale, NY (US); Nisha Palackal, White Plains, NY (US); Erica Pyles, New City, NY (US)

(21) Appl. No.: 18/640,720

(22) Filed: Apr. 19, 2024

Related U.S. Application Data
(60) Provisional application No. 63/464,277, filed on May 5, 2023.

(51) Int. Cl. G01N 33/569; G01N 21/76; G01N 33/543; G01N 33/58; G01N 33/68

(52) U.S. Cl. CPC G01N 33/56983; G01N 21/76; G01N 33/543; G01N 33/58; G01N 33/6854; G01N 2333/015; G01N 2333/908

(57) ABSTRACT

The present invention generally pertains to methods of testing for the presence of anti-drug antibodies (ADAs) against therapeutic viral vectors. In particular, the present invention pertains to the use of biotin-labeled AAV as a capture reagent and ruthenium-labeled AAV as a detection reagent for the detection and quantification of ADAs against AAVs.

### CROSS-REFERENCE TO RELATED APPLICATIONS

This application claims priority to and the benefit of U.S. Provisional Application No. 63/464,277, filed May 5, 2023 which is herein incorporated by reference.

### BACKGROUND

Adeno-associated viruses (AAVs) have been widely used as gene delivery vectors to deliver genetic material, such as delivering nucleic acids for gene therapy. Overall, AAV-based therapy has the advantages of being non-pathogenic and non-toxic, having cell type-specific infection, and offering different serotypes with varying cell transduction efficiencies. A disadvantage is that AAV production, purification, and characterization are more complex compared to, for example, antibody therapies.

While wild-type AAV is apparently a non-pathogenic virus, infection with wild-type AAV is common. 30-60% of all individuals may harbor pre-existing antibodies that may neutralize AAV transduction. Therefore, assays to detect and quantify pre-existing antibodies should be included in the assessment of clinical immunogenicity. There do not yet exist efficient methods for characterizing AAV anti-drug antibodies (ADAs) comparable to ADA assays for antibody therapeutics. ADA assays typically use a bridging format, using labeled drugs as both capture and detection reagents. For example, an ADA can bind both biotin-labeled and ruthenium-labeled drug, forming a bridge between the two. The bridged complex is captured by a streptavidin-coated plate surface. An electric current activates ruthenium-labeled drug to produce an electrochemiluminescent signal, which can be used to detect the presence of an ADA.

The relatively reduced number of accessible (surface-exposed) lysines compared to the size of an AAV presents unique challenges for lysine-directed conjugation of labels, and assays relying on labeled analytes, such as the ADA bridging assay. Furthermore, because of the large size of AAV, traditional methods of determining labeling efficiency, such as matrix-assisted laser desorption/ionization (MALDI), are not applicable.

Therefore, it will be appreciated that a need exists for methods and compositions for labeling AAV vectors, determining a degree of labeling of AAV vectors, and detecting anti-drug antibodies against AAV vectors.

### SUMMARY

This disclosure provides methods for detecting and/or quantifying an antibody against a viral capsid of interest in a sample. In some exemplary embodiments, the methods can comprise (a) contacting a sample to a capture reagent, a detection reagent, and a solid surface to form a detection mixture, wherein said capture reagent and said detection reagent are capable of binding to an antibody against a viral capsid of interest, and wherein said capture reagent is capable of binding to said solid surface; and (b) measuring said detection reagent to detect and/or quantify an antibody against said viral capsid of interest in said sample.

In one aspect, said sample is serum. In a specific aspect, said serum is human serum.

In one aspect, said capture reagent comprises biotin.

In one aspect, said capture reagent comprises said viral capsid of interest.

In one aspect, said detection reagent comprises a radiologic label, a photoluminescent label, a chemiluminescent label, a fluorescent label, a fluorophore, a hapten, an electrochemiluminescent label, or an enzyme label. In a specific aspect, said detection reagent comprises ruthenium or horseradish peroxidase.

In one aspect, said detection reagent comprises an antibody. In another aspect, said detection reagent comprises said viral capsid of interest.

In one aspect, said solid surface is selected from a group consisting of a microplate, resin, agarose beads, and magnetic beads. In another aspect, said solid surface is coated with avidin or streptavidin.

In one aspect, said antibody against said viral capsid of interest is an anti-drug antibody.

In one aspect, said viral capsid of interest is an AAV capsid. In a specific aspect, a serotype of said AAV capsid is AAV1, AAV2, AAV3, AAV4, AAV5, AAV6, AAV7, AAV8, AAVrh8, AAV2/8, AAV9, AAV10, AAV1, AAV12, a combination thereof, or a variant thereof. In another specific aspect, said AAV is a viral vector. In a more specific aspect, said AAV is a therapeutic viral vector.

In one aspect, said viral capsid is a nonenveloped viral capsid.

This disclosure also provides methods for detecting anti-drug antibodies against an AAV vector. In some exemplary embodiments, the methods can comprise (a) contacting serum to a capture reagent, a detection reagent, and a solid surface coated with avidin or streptavidin to form a detection mixture; (b) subjecting said detection mixture to an electrical current to produce a signal; and (c) measuring said signal to detect anti-drug antibodies against an AAV vector, wherein said capture reagent comprises said AAV vector conjugated to biotin and said detection reagent comprises said AAV vector conjugated to ruthenium.

In one aspect, said serum is human serum.

In one aspect, said solid surface is selected from a group consisting of a microplate, resin, agarose beads, and magnetic beads.

In one aspect, a serotype of said AAV vector is AAV1, AAV2, AAV3, AAV4, AAV5, AAV6, AAV7, AAV8, AAVrh8, AAV2/8, AAV9, AAV10, AAV1, AAV12, a combination thereof, or a variant thereof.

In one aspect, said AAV vector is a therapeutic vector.

In one aspect, said signal is an electrochemiluminescent signal.

This disclosure additionally provides compositions for detecting and/or quantifying an antibody against a viral capsid of interest in a sample. In some exemplary embodiments, the compositions can comprise (a) a sample; (b) a capture reagent; (c) a detection reagent; and (d) a solid surface, wherein said capture reagent and said detection reagent are capable of binding to an antibody against a viral capsid of interest, and wherein said capture reagent is capable of binding to said solid surface.

In one aspect, said sample is serum. In a specific aspect, said serum is human serum.

In one aspect, said capture reagent comprises biotin.

In one aspect, said capture reagent comprises said viral capsid of interest.

In one aspect, said detection reagent comprises a radiologic label, a photoluminescent label, a chemiluminescent label, a fluorescent label, a fluorophore, a hapten, an electrochemiluminescent label, or an enzyme label. In a specific aspect, said detection reagent comprises ruthenium or horseradish peroxidase.

In one aspect, said detection reagent comprises an antibody. In another aspect, said detection reagent comprises said viral capsid of interest.

In one aspect, said solid surface is selected from a group consisting of a microplate, resin, agarose beads, and magnetic beads. In another aspect, said solid surface is coated with avidin or streptavidin.

In one aspect, said antibody against said viral capsid of interest is an anti-drug antibody.

In one aspect, said viral capsid of interest is an AAV capsid. In a specific aspect, a serotype of said AAV capsid is AAV1, AAV2, AAV3, AAV4, AAV5, AAV6, AAV7, AAV8, AAVrh8, AAV2/8, AAV9, AAV10, AAV1, AAV12, a combination thereof, or a variant thereof.

In one aspect, said AAV is a viral vector. In a specific aspect, said AAV is a therapeutic viral vector.

In one aspect, said viral capsid is a nonenveloped viral capsid.

This disclosure further provides compositions for detecting anti-drug antibodies against an AAV vector. In some exemplary embodiments, the compositions can comprise (a) a sample; (b) an AAV vector conjugated to biotin; (c) an AAV vector conjugated to ruthenium; and (d) a solid surface coated with avidin or streptavidin, wherein said AAV vector conjugated to biotin and said AAV vector conjugated to ruthenium are modified forms of the same AAV vector.

In one aspect, said sample is serum. In a specific aspect, said serum is human serum.

In one aspect, said solid surface is selected from a group consisting of a microplate, resin, agarose beads, and magnetic beads.

In one aspect, a serotype of said AAV vector is AAV1, AAV2, AAV3, AAV4, AAV5, AAV6, AAV7, AAV8, AAVrh8, AAV2/8, AAV9, AAV10, AAV1, AAV12, a combination thereof, or a variant thereof.

In one aspect, said AAV vector is a therapeutic vector

This disclosure also provides methods for producing a labeled viral vector. In some exemplary embodiments, the methods can comprise (a) buffer exchanging a sample including a viral vector to produce a viral vector sample with basic pH; (b) contacting said viral vector sample with basic pH to N-hydroxysuccinimide ester linked to a label to produce a mixed sample including labeled viral vector; and (c) purifying said mixed sample to produce a labeled viral vector.

In one aspect, the labeled reagent was purified using Zeba Spin Desalting column 40K MWCO three times to remove any unlabeled dye.

In one aspect, said label is selected from a group consisting of a radiologic label, a photoluminescent label, a chemiluminescent label, a fluorescent label, a fluorophore, a hapten, an affinity label, an electrochemiluminescent label, and an enzyme label. In a specific aspect, said label is selected from a group consisting of biotin, ruthenium, and horseradish peroxidase.

In one aspect, said viral vector is an AAV vector. In a specific aspect, a serotype of said AAV vector is AAV1, AAV2, AAV3, AAV4, AAV5, AAV6, AAV7, AAV8, AAVrh8, AAV2/8, AAV9, AAV10, AAV1, AAV12, a combination thereof, or a variant thereof.

In one aspect, a concentration of said viral vector in said viral vector sample with basic pH is from about 10¹² to about 10¹³ vector genomes per mL.

In one aspect, a molar ratio of said N-hydroxysuccinimide ester linked to a label to said viral vector is from about 50:1 to about 20,000:1. In a specific aspect, said molar ratio is about 10,000:1.

In one aspect, said viral vector is a nonenveloped viral vector.

In one aspect, said purification comprises buffer exchanging said mixed sample to remove unconjugated label.

This disclosure additions provides methods for determining a degree of labeling of a viral capsid. In some exemplary embodiments, the methods can comprise (a) determining a concentration of conjugated label in a labeled viral capsid sample; (b) determining a concentration of viral capsid in said labeled viral capsid sample; and (c) dividing the concentration of (a) by the concentration of (b) to determine a degree of labeling of said viral capsid.

In one aspect, said viral capsid is an AAV capsid. In a specific aspect, a serotype of said AAV capsid is AAV1, AAV2, AAV3, AAV4, AAV5, AAV6, AAV7, AAV8, AAVrh8, AAV2/8, AAV9, AAV10, AAV1, AAV12, a combination thereof, or a variant thereof.

In one aspect, said label is selected from a group consisting of a radiologic label, a photoluminescent label, a chemiluminescent label, a fluorescent label, a fluorophore, a hapten, an affinity label, an electrochemiluminescent label, and an enzyme label. In a specific aspect, said label is ruthenium.

In one aspect, determining a concentration of conjugated label can comprise (a) subjecting said labeled viral capsid sample to size exclusion chromatography; (b) measuring light absorbance at a wavelength correspond to an absorption maximum of said label; (c) integrating the area under the curve corresponding to the main peak of the labeled viral capsid at the wavelength of (b) to determine a peak area; and (d) comparing said peak area to a standard curve relating peak area to concentration to determine a concentration of said conjugated label.

In one aspect, said wavelength is about 450 nm.

These, and other, aspects of the invention will be better appreciated and understood when considered in conjunction with the following description and accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1A shows a diagram of the monomer structures for the AAV VP proteins, with VP1 being the largest monomer, according to an exemplary embodiment.

FIG. 1B shows a comparison of different AAV serotypes and their percent identity, according to an exemplary embodiment.

FIG. 2 illustrates several analytical methods to characterize and monitor unique quality attributes of AAVs, according to an exemplary embodiment.

FIG. 3 shows a schematic of a traditional, bridging anti-drug antibody (ADA) assay format for monoclonal antibodies, according to an exemplary embodiment.

FIG. 4A shows a schematic of an ADA assay utilizing a biotin-labeled AAV as a capture reagent and an HRP-labeled anti-mouse IgG as a detection reagent, according to an exemplary embodiment.

FIG. 4B shows a schematic of an ADA assay utilizing a biotin-labeled AAV as a capture reagent and a ruthenium-labeled AAV as a detection reagent, according to an exemplary embodiment.

FIG. 5A shows the stability of AAV1 in buffers of varying pH as measured by polydispersity index (PDI), according to an exemplary embodiment.

FIG. 5B shows the stability of AAV8 in buffers of varying pH as measured by polydispersity index (PDI), according to an exemplary embodiment.

FIG. 6A illustrates an exemplary method for labeling AAV, including buffer exchange to a basic pH, challenge ratios of 100-10000×, and purification of the labeled AAVs, according to an exemplary embodiment.

FIG. 6B shows a size exclusion chromatography (SEC) chromatogram of successfully biotin-labeled and purified AAV, with biotin-labeled AAV measured at a retention time around 12.50 minutes and excess biotin measured at a retention time around 18.70 minutes, according to an exemplary embodiment.

FIG. 7A shows a schematic of an ADA assay utilizing a biotin-labeled AAV8 (capture reagent) forming a bridged molecule with mouse anti-AAV8 mAb and anti-mouse IgG-HRP (detection reagent), according to an exemplary embodiment.

FIG. 7B shows an effect of biotin-labeled AAV concentration on detected signal, utilizing both monoclonal and polyclonal antibodies from rabbit and mouse, according to an exemplary embodiment.

FIG. 7C shows signal produced from an ADA assay using normal human serum and biotin-labeled AAV8 as the capture reagent, according to an exemplary embodiment.

FIG. 8 shows an SEC chromatogram of successfully ruthenium-labeled and purified AAV, with ruthenium-labeled AAV measured at a retention time around 12.50 minutes and excess ruthenium measured at a retention time around 18.70 minutes according to an exemplary embodiment.

FIG. 9A illustrates an exemplary method for calculating a degree of labeling for a labeled AAV, according to an exemplary embodiment.

FIG. 9B shows a range of peak areas for ruthenium measured with 450 nm absorbance corresponding to a range of ruthenium concentrations, according to an exemplary embodiment.

FIG. 9C shows a standard curve relating the peak area for ruthenium measured with 450 nm absorbance to the concentration of ruthenium in μM, according to an exemplary embodiment.

FIG. 9D shows the calculated degree of labeling (DOL) of ruthenium-labeled monoclonal antibody compared to ruthenium-labeled AAV at two different challenge ratios (1,000:1 and 10,000:1), according to an exemplary embodiment.

FIG. 10A shows the amounts of excess ruthenium remaining after conjugation of ruthenium with an AAV and varying buffer exchange conditions, according to an exemplary embodiment.

FIG. 10B shows AAV and excess ruthenium recovered after conjugation of ruthenium with an AAV and varying buffer exchange conditions, according to an exemplary embodiment.

FIG. 11A shows signal from ADA bridging assays using biotin-labeled AAV8 as a capture reagent and ruthenium-labeled AAV8 as a detection reagent, at two different challenge ratios, according to an exemplary embodiment.

FIG. 11B shows signal from ADA bridging assays using biotin-labeled AAV8 as a capture reagent and ruthenium-labeled AAV8 as a detection reagent, at varying AAV8 concentrations and a 10,000:1 challenge ratio, according to an exemplary embodiment.

FIG. 12 shows signal from ADA bridging assays using biotin-labeled AAV8 as a capture reagent and ruthenium-labeled AAV8 as a detection reagent for normal human serum, at varying AAV8 concentrations, according to an exemplary embodiment.

### DETAILED DESCRIPTION

Adeno-associated viruses (AAVs) have been widely used as gene delivery vectors to deliver genetic material, such as delivering nucleic acids for gene therapy. AAVs provide the advantages of non-pathogenicity and low immunogenicity. AAVs are nonpathogenic members of the Parvoviridae family under Dependovirus genus and require helpers, such as Adenovirus or Herpesvirus, for infection (Venkatakrishnan et al., Structure and Dynamics of Adeno-Associated Virus Serotype 1 VP1—Unique N-Terminal Domain and Its Role in Capsid Trafficking, Journal of Virology, May 2013, vol. 87, no. 9, pages 4974-4984). AAV encapsulates a single-stranded DNA genome of about 4.8 kilobases (kb) in an icosahedral capsid which is made of a shell of capsid viral proteins. Recombinant AAV genomes are nonpathogenic and do not integrate into a host's genome, but exist as stable episomes that provide long-term expression. AAV serotypes make a very useful system for preferentially transducing specific cell types.

Overall, AAV-based therapy has the advantages of being non-pathogenic and non-toxic, having cell type-specific infection, and offering different serotypes with varying cell transduction efficiencies. A disadvantage is that AAV production, purification, and characterization are more complex compared to, for example, antibody therapies. Fully packaged AAVs consist of an icosahedral capsid containing an about 4.7 kb single-stranded genome. An empty capsid has a molecular weight of about 3750 kDa, while a full capsid with about 4.7 kb single-stranded genome has a molecular weight of about 5100 kDa. The purity of AAVs is defined by several product-related impurities, including empty capsids, capsids containing partial or incorrect genomes, and aggregated or degraded capsid.

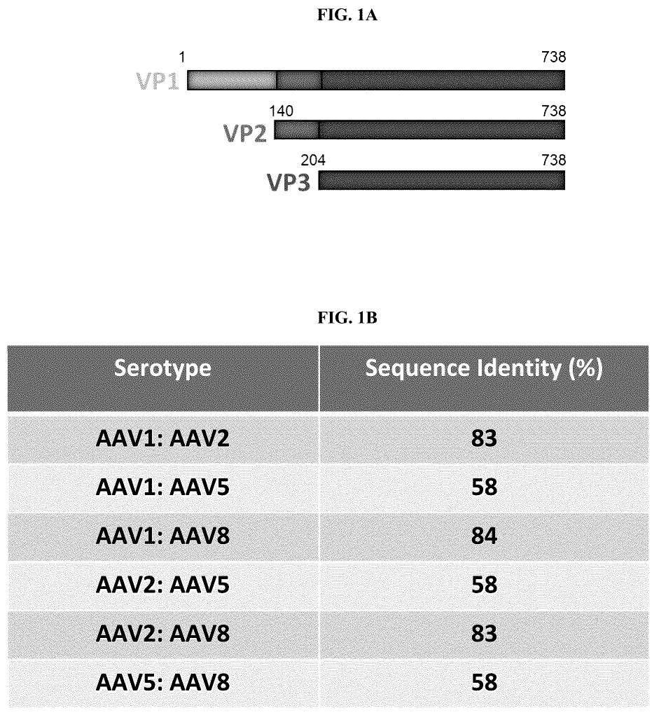

AAV capsids are composed of 60 subunits assembled from three proteins: VP1, VP2, and VP3, in proportions of approximately 1:1:10. These VP proteins have common C-termini, as shown in FIG. 1A. VP monomers form both trimers and pentamers that associate to form a full capsid. The core capsid structure is shared between different serotypes. The greatest variation in sequence is found in surface exposed loops. A comparison of sequence identity between serotypes is shown in FIG. 1B.

While wild-type AAV is apparently a non-pathogenic virus, infection with wild-type AAV is common. 30-60% of all individuals may harbor pre-existing antibodies that may neutralize AAV transduction. Therefore, assays to detect and quantify pre-existing antibodies should be included in the assessment of clinical immunogenicity. Immunogenicity of drug products, including viral vectors such as AAV vectors, is a major concern in clinical and preclinical studies, since it can lead to potentially serious side effects, loss of efficacy, and changes in drug exposure, complicating the interpretation of toxicity, pharmacokinetic (PK) and pharmacodynamics (PD) data.

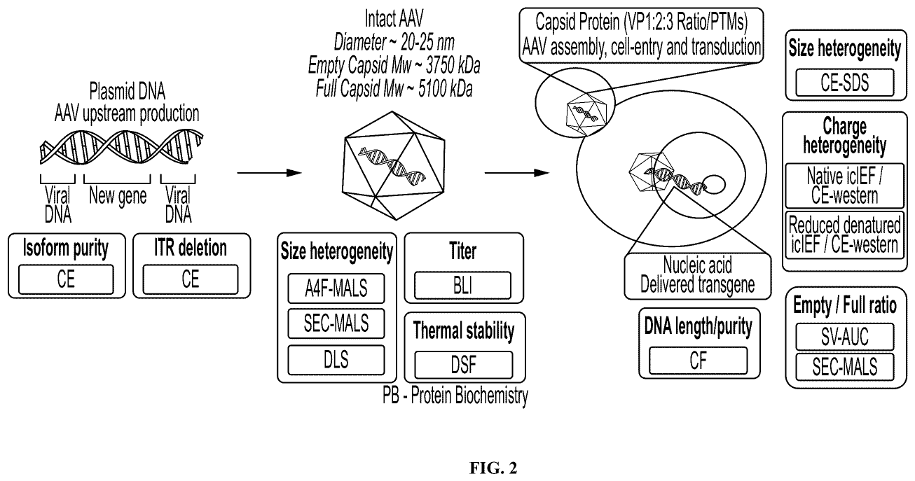

A variety of analytical methods have been developed to characterize and monitor unique quality attributes of AAVs, such as isoform purity, ITR deletion, size heterogeneity, charge heterogeneity, titer, thermal stability, and the empty/full ratio of capsids, as illustrated in FIG. 2. However, there do not yet exist efficient methods for characterizing AAV anti-drug antibodies (ADAs) comparable to ADA assays for antibody therapeutics. ADA immunoassays to detect and quantify ADAs are important in determining immunogenicity of biotherapeutics. ADA assays typically use a bridging format, using drugs as both capture and detection reagents. These assays are relatively easy to set up and run, detect most isotype responses, and provide excellent sensitivity. They are not species-specific and are high-throughput.

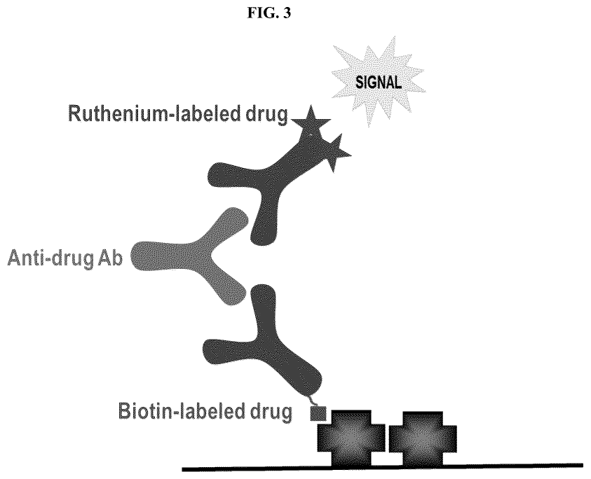

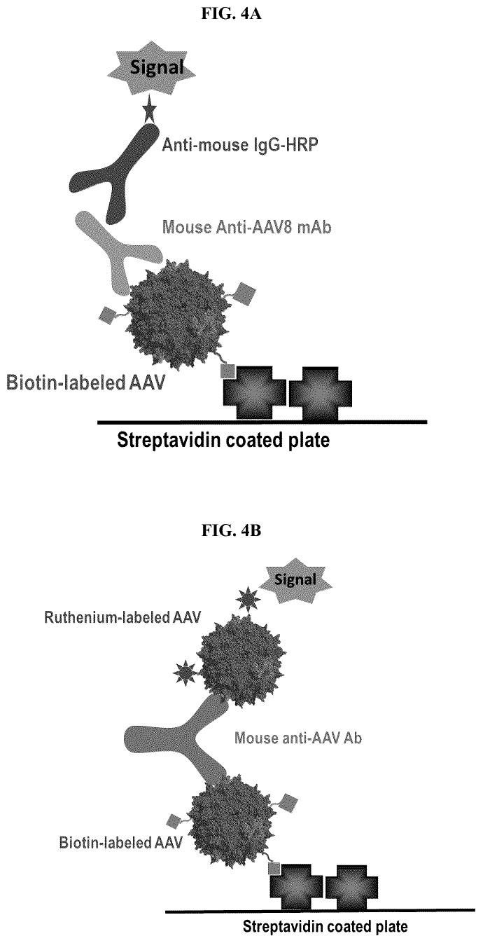

A traditional ADA format for monoclonal antibodies, commonly used throughout the industry, is shown in FIG. 3. Any ADA binds both biotin-labeled and ruthenium-labeled antibody drug, forming a bridge between the two. The bridged complex is captured by a streptavidin-coated plate surface. An electric current activates ruthenium-labeled drug to produce an electrochemiluminescent signal, which can be used to detect the presence of an ADA. The assay is highly sensitive and detects all ADA isotypes, and is useful for a non-quantitative, titer-based assessment of immunogenicity. Additional potential ADA assay formats, using the example of an AAV drug, are shown in FIG. 4A and FIG. 4B.

A comparison between an AAV and an antibody for the purposes of characterization and label conjugation is shown in Table 1. The relatively reduced number of accessible (surface-exposed) lysines compared to the size of an AAV presents unique challenges for lysine-directed conjugation of labels, and assays relying on labeled analytes, such as the ADA bridging assay. Furthermore, because of the large size of AAV, traditional methods of determining labeling efficiency, such as matrix-assisted laser desorption/ionization (MALDI), are not applicable.

TABLE 1
Physical characteristics of antibodies and AAVs

| | Antibody | AAV |
|---|---|---|
| Diameter (nm) | ~10 | 20-25 |
| Hydrodynamic radius (nm) | 5 | 12-18 |
| Molecular weight (Da) | ~145,000 | ~5,000,000 |
| Working concentration (M) | 6.9 × 10⁻⁵ | 3.3 × 10⁻⁸ |
| Accessible lysines | <40 | 300-500 |

As described above, there exists a need for methods and compositions for labeling AAV vectors for detection, and for detecting and quantifying anti-drug antibodies to therapeutic AAVs. This disclosure sets forth methods and compositions for labeling the capsids of AAV vectors, determining a degree of AAV labeling, and using labeled AAVs or AAV capsids to detect and quantify anti-drug antibodies to AAV vectors.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. “Synthetic peptides or polypeptides” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. A protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated). Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG (e.g., IgG1, IgG2, IgG3, IgG4), IgM, IgA1, IgA2, IgD, or IgE. In certain exemplary embodiments the antibody molecule is a full-length antibody (e.g., an IgG1 or IgG4 immunoglobulin) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated).

As used herein “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies can be prepared using a wide variety of techniques known in the art including the use of hybridoma, recombinant, and phage display technologies, or a combination thereof.

In some exemplary embodiments, a recombinant protein can be produced from mammalian cells. The mammalian cells can be of human origin or non-human origin, and can include primary epithelial cells (e.g., keratinocytes, cervical epithelial cells, bronchial epithelial cells, tracheal epithelial cells, kidney epithelial cells and retinal epithelial cells), established cell lines and their strains (e.g., 293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPMI2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK(15) cells, GHi cells, GH3 cells, L2 cells, LLC-RC 256 cells, MHiCi cells, XC cells, MDOK cells, VSW cells, and TH-I, B1 cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSA1 cells, Swiss/3T3 cells, Indian muntjac cells, SIRC cells, Cn cells, and Jensen cells, Sp2/0, NS0, NS1 cells or derivatives thereof).

As used herein, the term “anti-drug antibodies” or “ADAs” refers to antibodies produced by the immune system of a subject that target epitopes on a therapeutic protein or viral vector. The term “drug” should be understood to include monomeric proteins, multimeric proteins, small molecules, viral vectors, or any other such chemical entity produced for therapeutic or diagnostic purposes. Anti-drug antibodies may occur during therapy as an immunogenic reaction of a patient. In the case of viral vector therapies, patients may have ADAs due to prior exposure to a related virus. It should be understood that an ADA is not limited to an antibody produced as a response to a viral vector therapy, but includes antibodies that may occur in response to wild-type virus of the same or similar virus type or serotype as a viral vector of interest. For example, an antibody that may have been produced in response to a wild-type AAV8 infection would be considered an ADA in the context of an AAV8 viral vector, or any viral vector that could be bound by the antibody.

A subset of ADAs are “neutralizing antibodies” or “NAbs”, which can bind to a therapeutic protein, viral vector, or other drug in a manner that inhibits or neutralizes its pharmacological activity, for example by inhibiting transduction by a viral vector. NAbs may affect the clinical efficacy of a therapeutic protein or viral vector, and as such must be monitored when administering a therapeutic protein to a subject.

ADA immunoassays are well known to the skilled artisan. Methods for carrying out such assays as well as practical applications and procedures are well-known in the art and described in, for example, Colowick, S. P. and Caplan, N. O. (eds.), “Methods in Enzymology”, Academic Press, dealing with immunological detection methods, especially volumes 70, 73, 74, 84, 92, and 121. The principles of different immunoassays are described, for example, by Hage, D. S. (Anal. Chem. 71 (1999) 294R-304R). Lu, B., et al. (Analyst 121 (1996) 29R-32R) which describes the orientated immobilization of antibodies for the use in immunoassays. Avidin-biotin-mediated immunoassays are described, for example, by Wilchek, M., and Bayer, E. A., in Methods Enzymol. 184 (1990) 467-469.

A commonly used ADA assay method is a bridging immunoassay (sec, e.g., Liao, K., et al., J Immunol Methods, 2017, 441: p. 15-23; Dai, S., et al., AAPS J, 2014, 16(3): p. 464-77; and Zhong, Z. D., et al., AAPS J, 2017. 19(6): p. 1564-1575, the contents of which are incorporated by reference herein). An ADA bridging immunoassay is a sandwich-type immunoassay in which a multi-valent ADA is bound by a capture reagent and a detection reagent, optionally wherein one or both of the reagents are the drug of interest, each binding to a different, not overlapping or interfering epitope of the ADA. The capture reagent and/or the detection reagent may be a drug or therapeutic protein or vector of interest, or may be an antibody targeted against the ADA. In particular, in this assay, a sample is incubated with a capture reagent and a detection reagent, comprising a detectable label. After sample incubation, a sandwich comprising the capture reagent, the ADA, and the detection reagent is formed and, thus, the ADA bridges two reagents binding to it and the bound ADA can be detected. In one aspect, the immunoassay is a high-throughput assay.

The ADA bridging immunoassay further comprises determining the presence of or amount of an ADA. Thus, the present disclosure provides a detection reagent, for example an antibody, an AAV vector or AAV capsid, conjugated to a detectable label. Non-limiting examples of detectable labels for any of the methods of the invention include ruthenium, a radiologic label, a photoluminescent label, a chemiluminescent label, a fluorescent label, a fluorophore, a hapten, an electrochemiluminescent label, or an enzyme label. The detectable label can be measured using instruments and devices known to those skilled in the art.

This disclosure generally describes assays wherein a signal is generated by binding of an ADA to a drug, for example a viral vector. Assays wherein a signal is inhibited or quenched by binding of an ADA to a drug are also contemplated. For simplicity, this disclosure discusses assays wherein a signal results from binding of an ADA to a drug or other reagent, although the methods and compositions described herein may equally be applied to assays wherein a signal is inhibited or quenched by binding of an ADA to a drug or other reagent.

As used herein, the term “capture reagent” refers to a chemical entity that is capable of binding an antibody of interest, in particular an anti-drug antibody, and has affinity to a second target, in order to capture the antibody of interest. In some aspects, a capture reagent may be a biotinylated antibody, a biotinylated drug, or a biotinylated virus or viral capsid, which can then bind to a solid surface through an interaction between biotin and avidin or streptavidin. Other affinity tags suitable for use with a capture reagent are known in the art.

As used herein, the term “detection reagent” refers to a chemical entity that is capable of binding an antibody of interest, in particular an anti-drug antibody, and features a detectable label, in order to render the antibody of interest detectable for analysis. In some aspects, a detection reagent may be an antibody, a virus, or a viral capsid conjugated to HRP or ruthenium. Other detectable labels suitable for use with a detection reagent are known in the art and further described in this disclosure.

As used herein, an entity or reagent that is modified by the term “labeled” includes any entity that is conjugated with another molecule or chemical entity that is empirically detectable (a “detectable label”). Chemical species suitable as labels include, but are not limited to, ruthenium, a radiological label, a photoluminescent label, a chemiluminescent label, a fluorescent label, an electrochemiluminescent label, an affinity label, quantum dots, or an optical dye label. Labels may also include, for example, biotin, Protein A, Protein G, or glutathione S-transferase (GST). These labels can be used to label capture reagents or detection reagents.

Representative fluorophores for use in the methods provided herein include, for example, green fluorescent protein, blue fluorescent protein, red fluorescent protein, fluorescein, fluorescein 5-isothiocyanate (FITC), cyanine dyes (Cy3, Cy3.5, Cy5, Cy5.5, Cy7), Bodipy dyes (Invitrogen) and/or Alexa Fluor dyes (Invitrogen), dansyl, Dansyl Chloride (DNS-C1), 5-(iodoacetamida)fluorescein (5-IAF, 6-acryloyl-2-dimethylaminona-phthalene (acrylodan), 7-nitrobenzo-2-oxa-1,3-diazol-4-yl chloride (NBD-Cl), ethidium bromide, Lucifer Yellow, rhodamine dyes (5-carboxyrhodamine 6G hydrochloride, Lissamine rhodamine B sulfonyl chloride, rhodamine-B-isothiocyanate (RITC (rhodamine-B-isothiocyanate), rhodamine 800); tetramethylrhodamine 5-(and 6-)isothiocyanate (TRITC)), Texas Red, sulfonyl chloride, naphthalamine sulfonic acids including but not limited to 1-anilinonaphthalene-8-sulfonic acid (ANS) and 6-(p-toluidinyl)naphthalen-e-2-sulfonic acid (TNS), Anthroyl fatty acid, DPH, Parinaric acid, TMA-DPH, Fluorenyl fatty acid, Fluorescein-phosphatidylethanolamine, Texas red-phosphatidylethanolamine, Pyrenyl-phophatidylcholine, Fluorenyl-phosphotidylcholine, Merocyanine 540, Naphtyl Styryl, 3,3′dipropylthiadicarbocyanine (diS-C3-(5)), 4-(p-dipentyl amino styryl)-1-methylpyridinium (di-5-ASP), Cy-3 lodo Acetamide, Cy-5-N-Hydroxysuccinimide, Cy-7-Isothiocyanate, IR-125, Thiazole Orange, Azure B, Nile Blue, Al Phthalocyanine, Oxaxine 1,4′,6-diamidino-2-phenylindole. (DAPI), Hoechst 33342, TOTO, Acridine Orange, Ethidium Homodimer, N(ethoxycarbonylmethyl)-6-methoxyquinolinium (MQAE), Fura-2, Calcium Green, Carboxy SNARF-6, BAPTA, coumarin, phytofiuors, Coronene, and metal-ligand complexes.

Haptens for use in the methods provided herein include, for example, digoxigenin and biotin.

Enzymes for use in the methods provided herein include, for example, alkaline phosphatase (AP), β-galactosidase, horse radish peroxidase (HRP), soy bean peroxidase (SBP), urease, β-lactamase and glucose oxidase.

The efficiency and extent of labeling of a capture reagent or a detection reagent can be affected based on the relative molar concentration of the label, or tag, and the target reagent. The label may functionalized, for example by linking to N-hydroxysuccinimide ester to promote lysine conjugation. In some aspects, a molar ratio of a label, for example biotin or ruthenium linked to N-hydroxysuccinimide ester, to a target reagent, for example an antibody, a viral capsid or a viral vector, may be from about 50:1 to about 20,000:1, from about 100:1 to about 15,000:1, from about 100:1, to about 10,000:1, from about 1,000:1, to about 10,000:1, from about 9,000:1 to about 11,000:1, above 100:1, above 200:1, above 500:1, above 1,000:1, above 2,000:1, above 5,000:1, above 9,000:1, above 10,000:1, about 100:1, about 200:1, about 500:1, about 1,000:1, about 2,000:1, about 3,000:1, about 4,000:1, about 5,000:1, about 6,000:1, about 7,000:1, about 8,000:1, about 9,000:1, about 9,500:1, about 10,000:1, about 10,500;1, about 11,000:1, about 12,000:1, about 13,000:1, about 14,000:1, about 15,000:1, or about 20,000:1.

In one aspect, the capture reagent, for example an AAV vector or AAV capsid, is conjugated to a solid surface. In one aspect, the conjugation of the capture reagent to the solid surface is performed via a specific binding pair, wherein the capture reagent is labeled or conjugated. In one aspect, the specific binding pair (first component/second component) is selected from streptavidin or avidin/biotin, biotin/neutravidin, biotin/captavidin, antibody/antigen (sec, e.g., Hermanson, G. T., et al., Bioconjugate Techniques, Academic Press, 1996), epitope/antibody, protein A/immunoglobulin, protein G/immunoglobulin, protein L/immunoglobulin, GST/glutathione, His-tag/Nickel, FLAG/M1 antibody, maltose binding protein/maltose, calmodulin binding protein/calmodulin, enzyme/enzyme substrate, lectin/polysaccharide, steroid/steroid binding protein, hormone/hormone receptor, and receptor-ligand binding pairs. In one aspect, the capture reagent is conjugated to biotin (as first component of a specific binding pair). In this case the conjugation to the solid phase is performed via immobilized avidin or streptavidin.

In one aspect, a sample being tested in an ADA immunoassay is a serum sample. In one aspect, the serum is human serum.

In one aspect, incubation of the sample, the capture reagent and the detection reagent is carried out at room temperature. In one aspect, the incubation time of the sample, the capture reagent and the detection reagent is at least 0.5 hours. In another aspect, the incubation time is at least 1 hours. In one aspect the incubation time is at least 1.5 hours. In one aspect, the incubation time is up to 2 hours. In still another aspect, the incubation time is between 0.5 hours and 12 hours. In one aspect, the incubation time is between 0.5 hours and 5 hours. In another aspect, the incubation time is between 1 hours and 12 hours. In one aspect, the incubation time is between 1 hour and 5 hours. In another aspect, the incubation time is between 5 and 12 hours.

In one aspect, following incubation of the sample, the capture reagent and the detection reagent, the sample is transferred to a labeled solid surface, e.g., a streptavidin labeled solid surface, and incubated further, so that the capture reagent can attach to the solid surface. In one aspect, incubation is at room temperature. In another aspect, following incubation the samples are analyzed for binding to an ADA using any method known in the art for detection of a detectable label, for example ruthenium. A true positive signal in the ADA bridging assay results from bivalent binding of the ADA to the capture reagent and the detection reagent, forming a bridge.

As used herein, the term “solid surface” refers to a non-fluid substance, and includes particles (including microparticles and beads) made from materials such as polymer, metal (paramagnetic, ferromagnetic particles), glass, and ceramic; gel substances such as silica, alumina, and polymer gels; capillaries, which may be made of polymer, metal, glass, and/or ceramic; zeolites and other porous substances; electrodes; microtiter plates; solid strips; cuvettes, tubes, or other containers. Solid surfaces for the immunoassays described herein are widely described in the state of the art (sec, e.g., Butler, J. E., Methods 22 (2000) 4-23, which is incorporated herein by reference). A solid surface component of an assay is distinguished from inert solid surfaces with which the assay may be in contact in that a “solid surface” contains at least one moiety on its surface, which is intended to interact with the capture drug. A solid surface may be a stationary component, such as a tube, strip, cuvette, or microtiter plate, or may be a non-stationary component, such as beads and microparticles. Microparticles can also be used as a solid phase for homogeneous surface formats. A variety of microparticles that allow either non-covalent or covalent attachment of proteins and other substances may be used. Such particles include polymer particles such as polystyrene and poly(methylmethacrylate); gold particles such as gold nanoparticles and gold colloids; and ceramic particles such as silica, glass, and metal oxide particles. See, e.g., Martin, C. R., et al., Analytical Chemistry—News & Features 70 (1998) 322A-327A, which is incorporated herein by reference.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the nucleic acid can comprise sugars and phosphate groups (as may typically be found in RNA or DNA), or modified or substituted sugar or phosphate groups.

Alternatively, the backbone of the nucleic acid can comprise a polymer of synthetic subunits such as phosphoramidates and thus can be an oligodeoxynucleoside phosphoramidate (P—NH2) or a mixed phosphoramidate-phosphodiester oligomer. In addition, a double-stranded nucleic acid can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

“Adeno-associated virus” or “AAV” is a non-pathogenic parvovirus, with single-stranded DNA, a genome of approximately 4.7 kb, not enveloped, and with an icosahedric conformation. AAV was first discovered in 1965 as a contaminant of adenovirus preparations. AAV belongs to the Dependovirus genus and Parvoviridae family, requiring helper functions from either herpes virus or adenovirus for replication. In the absence of helper virus, AAV can set up latency by integrating into human chromosome 19 at the 19q13.4 location. The AAV genome consists of two open reading frames (ORF), one for each of two AAV genes, Rep and Cap. The AAV DNA ends have a 145-bp inverted terminal repeat (ITR), and the 125 terminal bases are palindromic, leading to a characteristic T-shaped hairpin structure.

The Rep gene is transcribed from promoters p5 and p19 into four Rep proteins (Rep78, Rep68, Rep52, and Rep40), which have important roles in the life cycle of the virus. Proteins Rep78 and Rep68 are encoded by the mRNA transcribed from promoter p5. These proteins are essential for viral DNA replication, transcription and control of site-specific integration. The two smaller proteins Rep52 and Rep40 are generated by the mRNA transcribed from promoter p19. These proteins are involved in the formation of a single-stranded viral genome for packaging and viral integration. The Cap gene encodes three viral capsid proteins: VP1 (735 amino acids, ^{˜}90 kDa), VP2 (598 amino acids, ^{˜}72 kDa) and VP3 (533 amino acids, ^{˜}60 kDa), which form the viral capsid of 60 subunits, at the ratio of 1:1:10. The three capsid proteins are translated from the mRNA transcribed from the promoter p40.

The terms “enveloped,” “nonenveloped,” “not enveloped” and the like refer to the viral envelope, a lipid bilayer surrounding the capsid of some viruses. The methods and compositions of the present disclosure are not limited to enveloped or nonenveloped viruses.

A “recombinant AAV vector (rAAV vector)” refers to a polynucleotide vector including one or more heterologous sequences (i.e., nucleic acid sequence not of AAV origin) that may be flanked by at least one, e.g., two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins).

A “capsid” is the protein shell of a virus, which encloses the genetic material. Three viral capsid proteins, VP1, VP1 and VP3, form the viral icosahedral capsid of 60 subunits in a ratio of 1:1:10. A full capsid contains genetic material and is required to provide therapeutic benefit. An empty capsid lacks the genome and therefore lack the ability to provide therapeutic benefit to the patient.

A “viral particle” refers to a viral particle composed of at least one viral capsid protein and an encapsulated viral genome. While AAV is described in this disclosure as a model virus or viral particle, it is contemplated that the disclosed methods can be applied to profile a variety of viruses, e.g., the viral families, subfamilies, and genera. In some aspects, the viral capsid, virus, or viral particle belongs to a viral family selected from the group consisting of Adenoviridae, Parvoviridae, Retroviridae, Baculoviridae, and Herpesviridae. In some aspects, the viral capsid, virus, or viral particle belongs to a viral genus selected from the group consisting of Atadenovirus, Aviadenovirus, Ichtadenovirus, Mastadenovirus, Siadenovirus, Ambidensovirus, Brevidensovirus, Hepandensovirus, Iteradensovirus, Penstyldensovirus, Amdoparvovirus, Aveparvovirus, Bocaparvovirus, Copiparvovirus, Dependoparvovirus, Erythroparvovirus, Protoparvovirus, Tetraparvovirus, Alpharetrovirus, Betaretrovirus, Deltaretrovirus, Epsilonretrovirus, Gammaretrovirus, Lentivirus, Spumavirus, Alphabaculovirus, Betabaculovirus, Deltabaculovirus, Gammabaculovirus, Iltovirus, Mardivirus, Simplexvirus, Varicellovirus, Cytomegalovirus, Muromegalovirus, Proboscivirus, Roseolovirus, Lymphocryptovirus, Macavirus, Percavirus, and Rhadinovirus.

For the purposes of detecting, quantifying and/or characterizing antibodies against a viral vector, it should be understood that an antibody that binds a viral vector typically will bind to the capsid of the viral vector. Therefore, descriptions of an antibody binding a viral capsid, viral vector, virus, viral particle, and the like, should be understood to be generally describing the same antibody-capsid binding, and therefore an assay using a viral capsid, viral vector, virus, viral particle and the like as a capture reagent and/or detection reagent for antibodies may be expected to identify, characterize, or quantify substantially the same antibodies.

“Heterologous” means derived from a genotypically distinct entity from that of the rest of the entity to which it is compared or into which it is introduced or incorporated. For example, a nucleic acid introduced by genetic engineering techniques into a different cell type is a heterologous nucleic acid (and, when expressed, can encode a heterologous polypeptide). Similarly, a cellular sequence (e.g., a gene or portion thereof) that is incorporated into a viral vector is a heterologous nucleotide sequence with respect to the vector. A heterologous nucleic acid that encodes a polypeptide may be referred to as a transgene.

An “inverted terminal repeat” or “ITR” sequence is relatively short sequences found at the termini of viral genomes which are in opposite orientation. An “AAV inverted terminal repeat (ITR)” sequence, is an approximately 145-nucleotide sequence that is present at both termini of a single-stranded AAV genome.

As used herein, the term “vector” refers to a recombinant plasmid or virus (“viral vector”) that comprises a nucleic acid to be delivered into a host cell, either in vitro or in vivo. Vectors derived from AAV are particularly attractive for delivering genetic material because (i) they are able to infect (transduce) a wide variety of non-dividing and dividing cell types including muscle fibers and neurons; (ii) they are devoid of the virus structural genes, thereby eliminating the natural host cell responses to virus infection, for example, interferon-mediated responses; (iii) wild type AAVs have never been associated with any pathology in humans; (iv) in contrast to wild type AAVs, which are capable of integrating into the host cell genome, replication-deficient AAV vectors generally persist as episomes, thus limiting the risk of insertional mutagenesis or activation of oncogenes; and (v) in contrast to other vector systems, AAV vectors do not trigger a significant immune response (see ii), thus granting long-term expression of the therapeutic transgenes (provided their gene products are not rejected).

A “recombinant viral vector” refers to a recombinant polynucleotide vector including one or more heterologous sequences (i.e., nucleic acid sequence not of viral origin).

In some aspects, a sample is a biological sample. As used herein, the term “biological sample” refers to a sample taken from a living organism, for example a human or non-human mammal. A biological sample may comprise or consist of, for example, whole blood, plasma, serum, saliva, tears, semen, check tissue, organ tissue, urine, feces, skin, or hair. A sample may be taken from a patient, for example, a clinical sample. In some exemplary embodiments, a sample may be taken from a non-human animal, for example, a preclinical sample. In some embodiments, a sample is a further processed form of any of the aforementioned examples of samples. A sample may comprise an antibody against a drug of interest, for example a viral vector, an AAV vector, or specifically the capsid thereof. The antibody may be an antibody produced by the immune system of a patient or non-human animal, or it may be a recombinant antibody.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reversed phase (RP) liquid chromatography, ion-exchange (IEX) chromatography, size exclusion chromatography (SEC), affinity chromatography, hydrophobic interaction chromatography (HIC), hydrophilic interaction chromatography (HILIC), or mixed-mode chromatography (MMC).

In some aspects, the methods of the present invention include the use of size exclusion chromatography. Size exclusion chromatography or gel filtration relies on the separation of components as a function of their molecular size. Separation depends on the amount of time that the substances spend in the porous stationary phase as compared to time in the fluid. The probability that a molecule will reside in a pore depends on the size of the molecule and the pore. In addition, the ability of a substance to permeate into pores is determined by the diffusion mobility of macromolecules which is higher for small macromolecules. Very large macromolecules may not penetrate the pores of the stationary phase at all; and, for very small macromolecules the probability of penetration is close to unity. While components of larger molecular size move more quickly past the stationary phase, components of small molecular size have a longer path length through the pores of the stationary phase and are thus retained longer in the stationary phase.

The chromatographic material can comprise a size exclusion material wherein the size exclusion material is a resin or membrane. The matrix used for size exclusion is preferably an inert gel medium which can be a composite of cross-linked polysaccharides, for example, cross-linked agarose and/or dextran in the form of spherical beads. The degree of cross-linking determines the size of pores that are present in the swollen gel beads. Molecules greater than a certain size do not enter the gel beads and thus move through the chromatographic bed the fastest. Smaller molecules, such as detergent, protein, DNA and the like, which enter the gel beads to varying extent depending on their size and shape, are retarded in their passage through the bed. Molecules are thus generally eluted in the order of decreasing molecular size.

Porous chromatographic resins appropriate for size-exclusion chromatography of viruses may be made of dextrose, agarose, polyacrylamide, or silica which have different physical characteristics. Polymer combinations can also be also used. Most commonly used are those under the tradename “SEPHADEX” available from Amersham Biosciences. Other size exclusion supports from different materials of construction are also appropriate, for example Toyopearl 55F (polymethacrylate, from Tosoh Bioscience, Montgomery Pa.) and Bio-Gel P-30 Fine (BioRad Laboratories, Hercules, Calif.).

In some aspects, the mobile phase used to obtain said eluate from size exclusion chromatography can comprise a volatile salt. In some specific aspects, the mobile phase can comprise ammonium acetate, ammonium bicarbonate, or ammonium formate, or combinations thereof.

It is understood that the present invention is not limited to any of the aforesaid protein(s), antibody(ies), ADA assay(s), capture reagent(s), detection reagent(s), label(s), sample(s), serotype(s), vector(s), or liquid chromatography system(s), and any protein(s), antibody(ies), ADA assay(s), capture reagent(s), detection reagent(s), label(s), sample(s), serotype(s), vector(s), or liquid chromatography system(s) can be selected by any suitable means.

The present invention will be more fully understood by reference to the following Examples. They should not, however, be construed as limiting the scope of the invention.

### EXAMPLES

### Example 1. Development of Biotin-Labeled AAVs

As described above, there exists a need for methods for labeling and characterizing AAVs, for example for characterizing ADAs to therapeutic AAVs. As a first step in developing a diagnostic assay for therapeutic AAVs, methods for labeling AAV molecules using biotin and ruthenium were evaluated. An approach of lysine-directed conjugation was investigated.

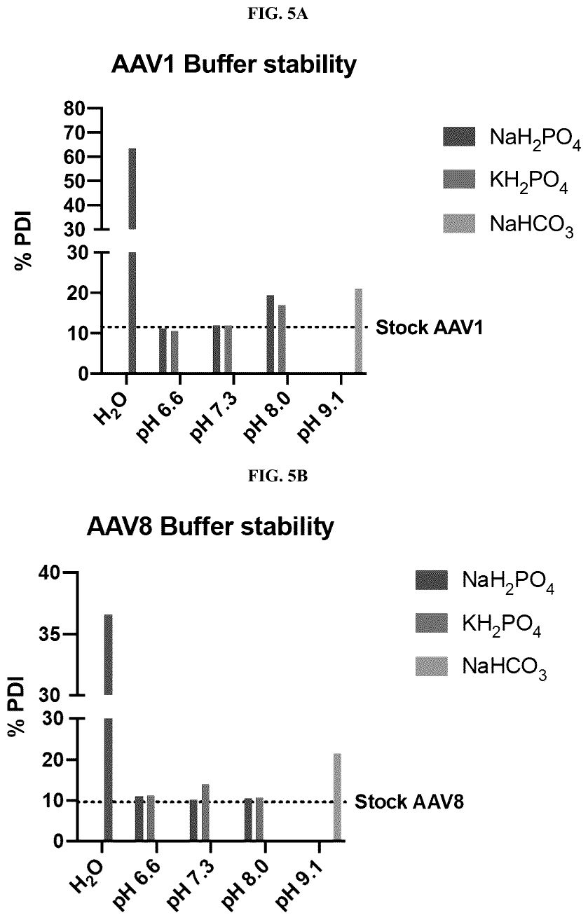

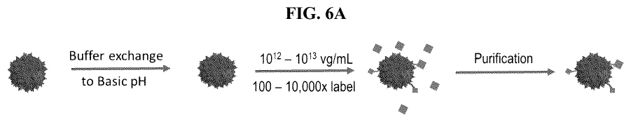

The stability of AAVs in buffers amenable to lysine-directed conjugation was investigated. AAV1 and AAV8 were stored in various buffers with pH up to 8.0, including water, NaH₂PO₄, KH₂PO₄, and NaHCO₃, and stability was measured. In particular, the polydispersity index (PDI), which is a measure of size heterogeneity of a sample, was monitored. PDI values of less than 20% were considered acceptable. Both serotypes of AAVs were found to be stable in the buffers tested, as shown in FIG. 5A and FIG. 5B. 20 mM buffers were used. Various ionic strengths (0, 20, or 200 mM additional NaCl or KCl) were tested, and no effect on PDI was found.

Therefore, lysine-directed conjugation with biotin was tested. Lysine-directed conjugation includes incubation of a protein with N-hydroxysuccinimide (NHS) ester, which allows crosslinking with primary amines (primarily, lysines). An NHS ester can be linked to a tag (or “label”), for example biotin or ruthenium, which will cause the tag to be covalently bound to a lysine.

Challenges of conjugating AAVs include the limited amount of AAV material at low concentrations; the potential for aggregation and/or low recovery when concentrating and purifying; and the limited available information for AAV conjugations, which demanded an empirical and inventive approach to successfully producing effectively labeled AAV.

Parameters investigated for AAV conjugation included the ratio of moles of tag molecule to moles of protein in the mixture (the challenge ratio), the number of available lysines, the reaction conditions (for example, pH and incubation time), the solvent for NHS ester, the AAV buffer formulation, and whether AAVs were full or empty.

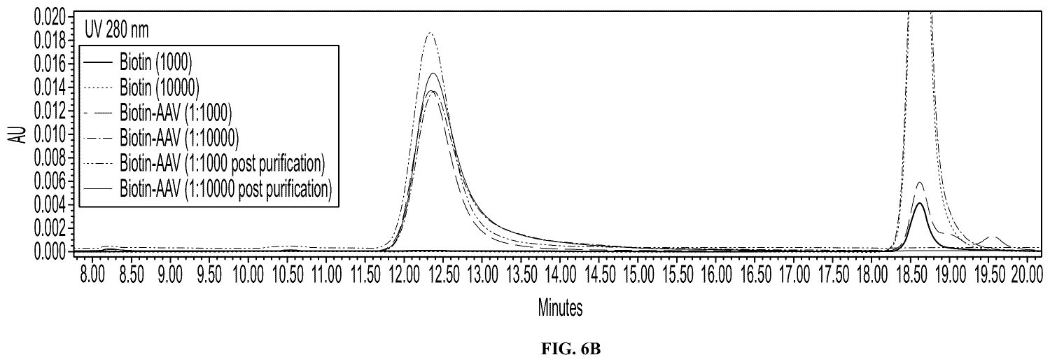

An exemplary procedure for conjugation of AAVs with biotin is shown in FIG. 6A, including buffer exchange to a basic pH, challenge ratios of 100-10000×, and purification of the labeled AAVs. Following successful conjugation, biotin-conjugated AAV8 was successfully purified and characterized using SEC, as shown in FIG. 6B.

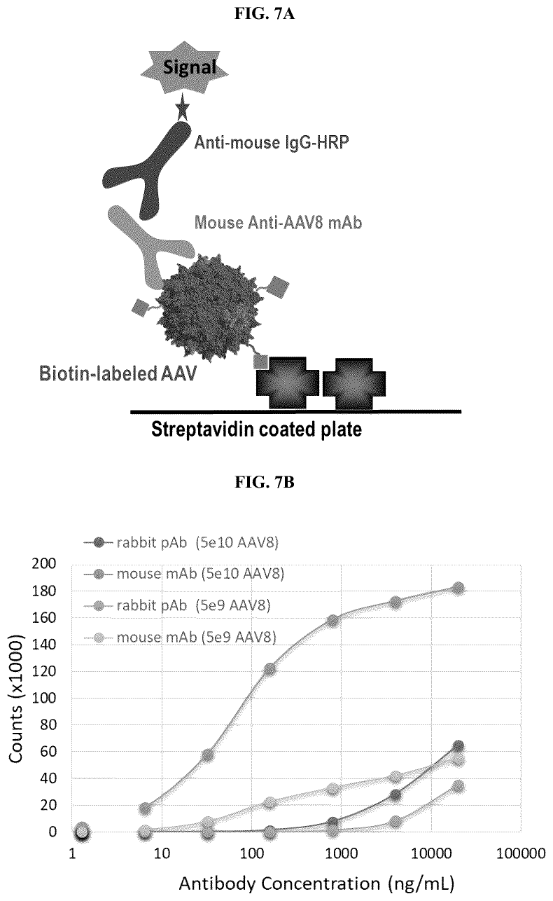

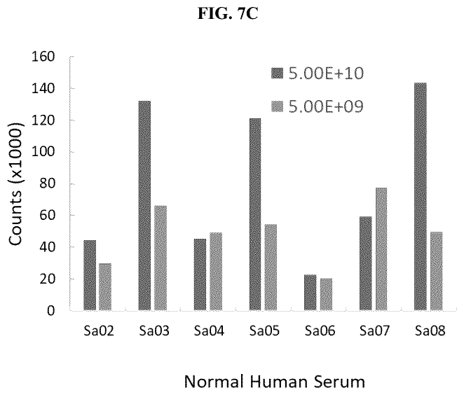

Biotinylated AAV was subjected to an anti-drug antibody (ADA) assay in the direct assay format, with mouse anti-AAV8 mAb used as a model for an anti-drug antibody, as shown in FIG. 7A. Biotin-labeled AAV was used as a capture reagent and HRP-labeled anti-mouse IgG was used as a detection reagent. Biotinylated AAV at higher concentrations produced a good signal to noise ratio in the ADA assay, as shown in FIG. 7B. The direct assay format produced more non-specific signals when used to assess normal human serum, as shown in FIG. 7C. Therefore, a traditional assay format using labeled AAV to produce a measurable signal, or in other words using a labeled drug as a detection reagent, was pursued.

### Example 2. Development of Ruthenium-Labeled AAVs and Determination of DOL

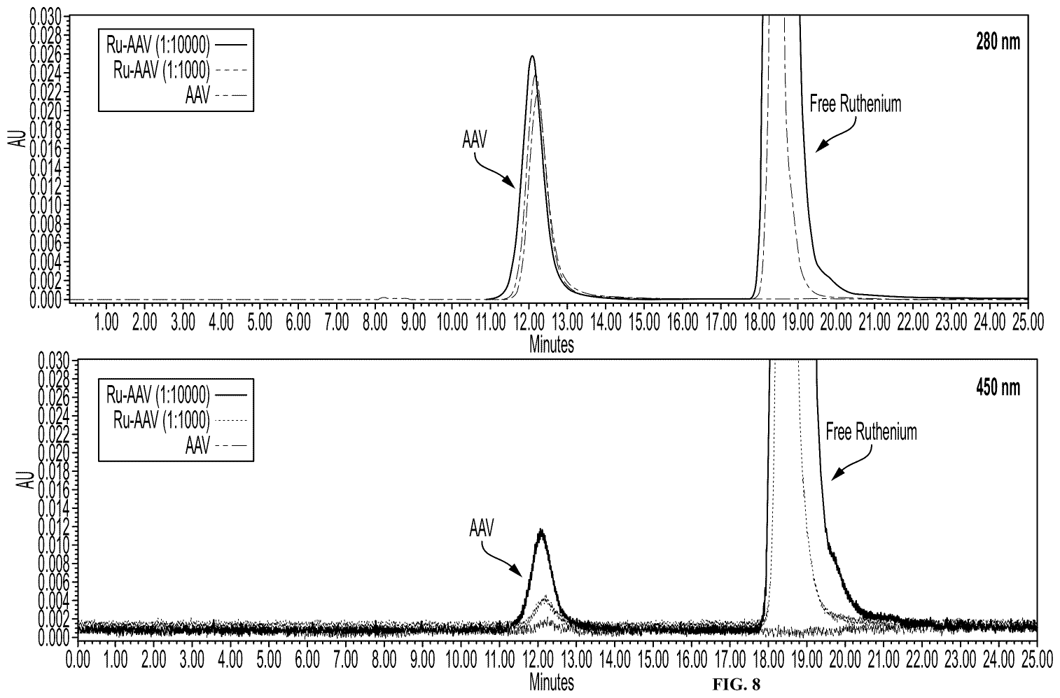

Ruthenium-labeled AAV8 was generated, using the general process described above for biotin conjugation, using high challenge ratios. MSD® TAG-NHS Ester (Ruthenium (II) tris-bipyridine, N-hydroxysuccinimide) from Meso Scale Discovery was used for conjugating the ruthenium label. Labeled AAV concentration and degree of labeling (DOL) were determined using size exclusion chromatography (SEC), as shown in FIG. 8. Protein absorption was measured at 280 nm wavelength and tag absorption was measured at 450 nm wavelength. The absorption maximum of the ruthenium tag is about 455 nm.

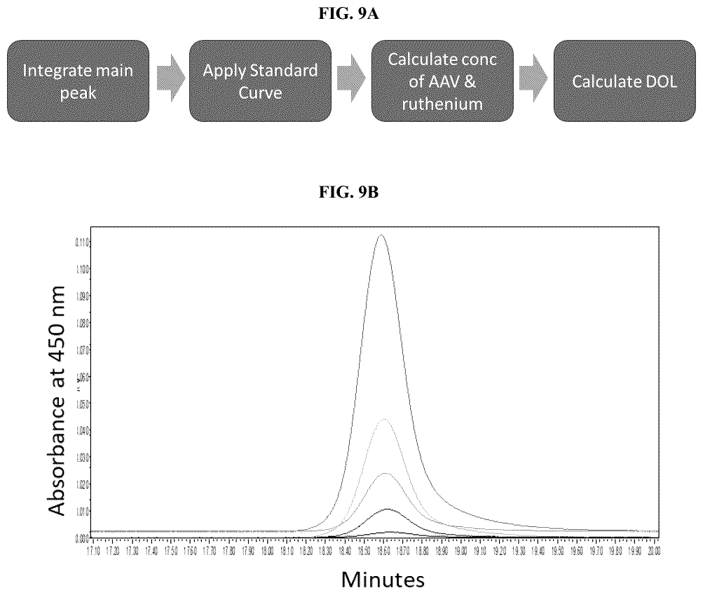

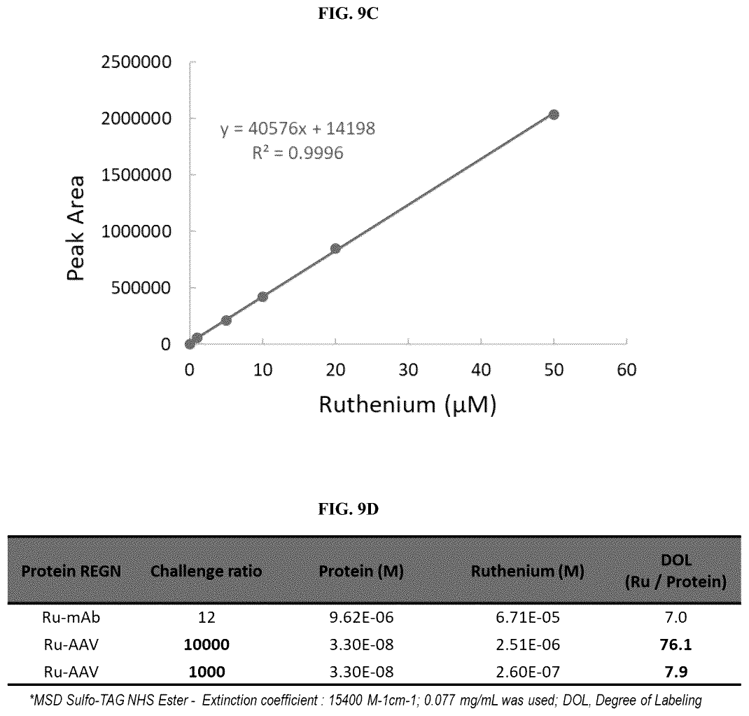

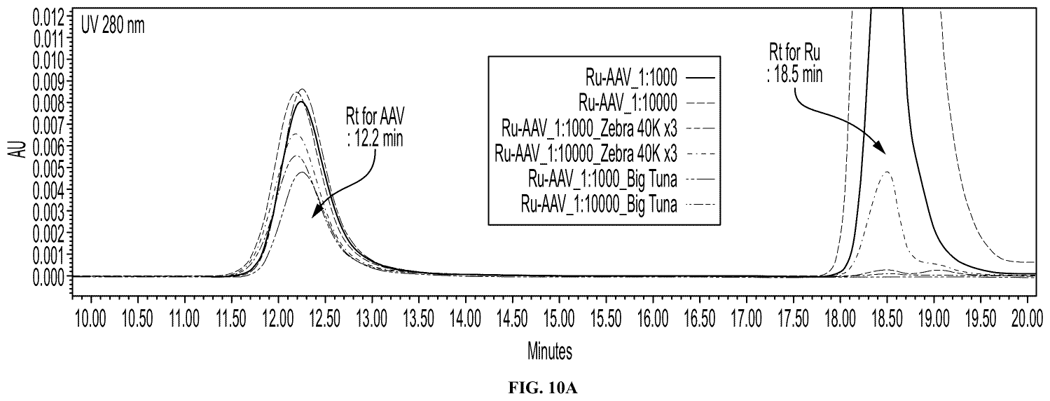

An exemplary method for calculating DOL is shown in FIG. 9A. The area under the curve of the main AAV peak was integrated and compared to a standard curve in order to calculate a concentration of AAV and of ruthenium. The concentration of ruthenium over the concentration of AAV was used as the DOL.

A standard curve was generated by analyzing various concentrations of ruthenium using SEC with absorbance measured at 450 nm, integrating the area under the curve (peak area), and deriving an equation relating the peak area to known concentration, as shown in FIG. 9C.

A comparison of ruthenium labeling of an exemplary mAb and AAV is shown in FIG. 9D. A significantly higher challenge ratio was needed to successfully label AAVs compared to mAbs.

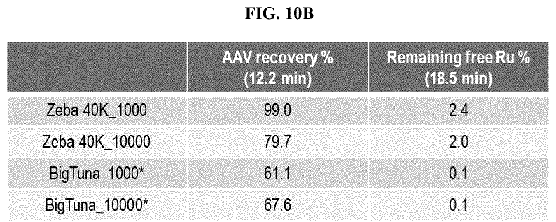

Methods for purifying the labeled AAV sample, for example removing unbound ruthenium, were optimized. Following a labeling reaction between AAV8 (2.57×10¹³ vg/mL) and MSD® TAG-NHS Ester, with a challenge ratio of 1,000:1 or 10,000:1, labeled AAV8 was optionally subjected to a buffer exchange step, and then recovery of AAV and free ruthenium was assessed using SEC, with absorbance measured at 280 nm and 450 nm. Two buffer exchange systems were compared: the Big Tuna buffer exchange platform (Unchained Labs) and Zeba™ Spin Desalting Columns (Thermo Scientific) with 40K MWCO (molecular weight cut-off). The Big Tuna platform was found to have high efficiency for removal of free ruthenium, as shown in FIG. 10A and FIG. 10B.

### Example 3. Development of an ADA Bridging Assay for Therapeutic AAV

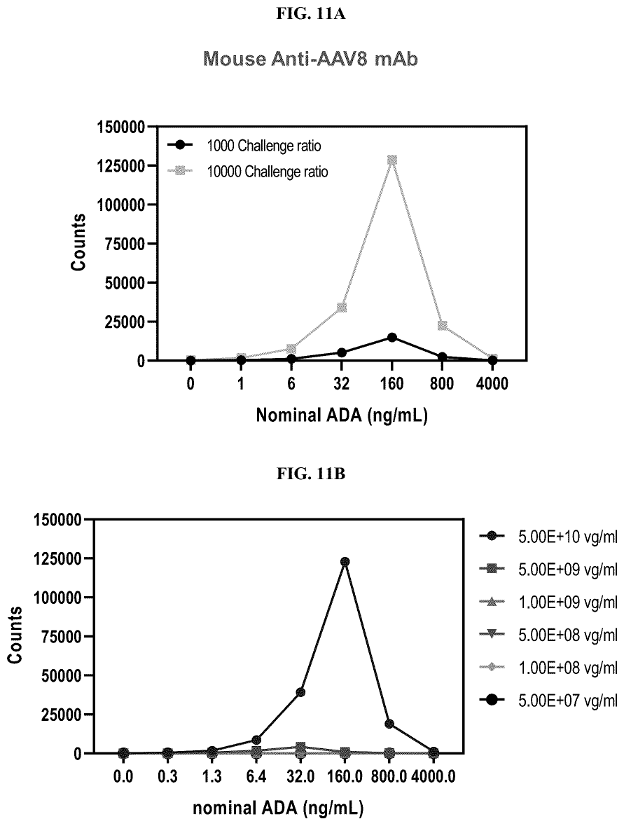

Biotin-labeled and ruthenium-labeled AAV8 produced as described above were used in an ADA bridging assay, using mouse anti-AAV8 antibody as a model for an ADA, as shown in FIG. 4B. High sensitivity was achieved in the ADA bridging assay with higher challenge ratios, as shown in FIG. 11A, and higher AAV concentrations, as shown in FIG. 11B.

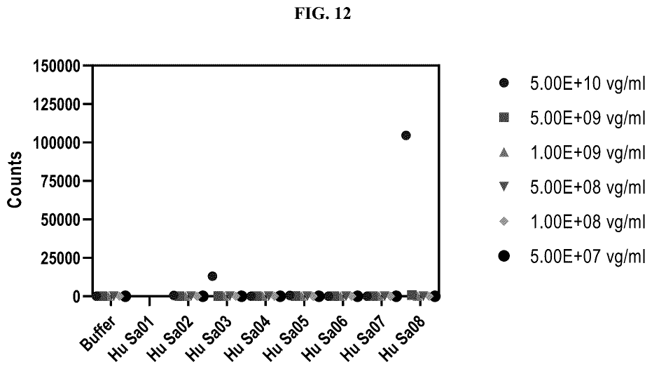

The novel ADA bridging assay for AAVs was tested using human serum. Anti-AAV8 ADAs were detected in a normal human serum sample at 1:10 dilution, as shown in FIG. 12.

In conclusion, a broad array of biochemical and biophysical methods were developed to characterize AAVs. AAVs were successfully labeled with biotin and demonstrated to work in a direct ADA binding assay. AAVs were also successfully labeled with ruthenium, and DOL was determined using SEC analysis. Compared to the previous state in the art for labeling therapeutic antibodies, labeling AAVs required novel approaches, including a challenge ratio of over 1,000:1 in order to produce labeled AAVs that were effective in assay development.

Biotin and ruthenium labeled AAVs were used to develop a sensitive and selective ADA bridging assay, which was capable of detecting ADAs in normal human serum diluted to 1:10. These findings provide a basis for methods of detecting labeled AAVs, and for diagnostic assays to support the clinical development of AAV programs, for example to assess immunogenicity for patient exclusion criteria.

What is claimed is:

1. A method for detecting and/or quantifying an antibody against a viral capsid of interest in a sample, comprising:
(a) contacting a sample to a capture reagent, a detection reagent, and a solid surface to form a detection mixture, wherein said capture reagent and said detection reagent are capable of binding to an antibody against a viral capsid of interest, and wherein said capture reagent is capable of binding to said solid surface; and
(b) measuring said detection reagent to detect and/or quantify an antibody against said viral capsid of interest in said sample.

2. The method of claim 1, wherein said sample is serum.

3. The method of claim 2, wherein said serum is human serum.

4. The method of claim 1, wherein said capture reagent comprises biotin.

5. The method of claim 1, wherein said capture reagent comprises said viral capsid of interest.

6. The method of claim 1, wherein said detection reagent comprises a radiologic label, a photoluminescent label, a chemiluminescent label, a fluorescent label, a fluorophore, a hapten, an electrochemiluminescent label, or an enzyme label.

7. The method of claim 6, wherein said detection reagent comprises ruthenium or horseradish peroxidase.

8. The method of claim 1, wherein said detection reagent comprises an antibody.

9. The method of claim 1, wherein said detection reagent comprises said viral capsid of interest.

10. The method of claim 1, wherein said solid surface is selected from a group consisting of a microplate, resin, agarose beads, and magnetic beads.

11. The method of claim 1, wherein said solid surface is coated with avidin or streptavidin.

12. The method of claim 1, wherein said antibody against said viral capsid of interest is an anti-drug antibody.

13. The method of claim 1, wherein said viral capsid of interest is an AAV capsid.

14. The method of claim 13, wherein a serotype of said AAV capsid is AAV1, AAV2, AAV3, AAV4, AAV5, AAV6, AAV7, AAV8, AAVrh8, AAV2/8, AAV9, AAV10, AAV1, AAV12, a combination thereof, or a variant thereof.

15. The method of claim 13, wherein said AAV is a viral vector.

16. The method of claim 15, wherein said AAV is a therapeutic viral vector.

17. The method of claim 1, wherein said viral capsid is a nonenveloped viral capsid.

18. A method for detecting anti-drug antibodies against an AAV vector, comprising:
(a) contacting serum to a capture reagent, a detection reagent, and a solid surface coated with avidin or streptavidin to form a detection mixture;
(b) subjecting said detection mixture to an electrical current to produce a signal; and
(c) measuring said signal to detect anti-drug antibodies against an AAV vector,
wherein said capture reagent comprises said AAV vector conjugated to biotin and said detection reagent comprises said AAV vector conjugated to ruthenium.

19. The method of claim 18, wherein said serum is human serum.

20. The method of claim 18, wherein said solid surface is selected from a group consisting of a microplate, resin, agarose beads, and magnetic beads.

21. The method of claim 18, wherein a serotype of said AAV vector is AAV1, AAV2, AAV3, AAV4, AAV5, AAV6, AAV7, AAV8, AAVrh8, AAV2/8, AAV9, AAV10, AAV1, AAV12, a combination thereof, or a variant thereof.

22. The method of claim 18, wherein said AAV vector is a therapeutic vector.

23. The method of claim 18, wherein said signal is an electrochemiluminescent signal.

24. A composition for detecting and/or quantifying an antibody against a viral capsid of interest in a sample, comprising:
(a) a sample;
(b) a capture reagent;
(c) a detection reagent; and
(d) a solid surface,
wherein said capture reagent and said detection reagent are capable of binding to an antibody against a viral capsid of interest, and wherein said capture reagent is capable of binding to said solid surface.

25. The method of claim 24, wherein said sample is serum.

26. The method of claim 25, wherein said serum is human serum.

27. The method of claim 24, wherein said capture reagent comprises biotin.

28. The method of claim 24, wherein said capture reagent comprises said viral capsid of interest.

29. The method of claim 24, wherein said detection reagent comprises a radiologic label, a photoluminescent label, a chemiluminescent label, a fluorescent label, a fluorophore, a hapten, an electrochemiluminescent label, or an enzyme label.

30. The method of claim 29, wherein said detection reagent comprises ruthenium or horseradish peroxidase.

31. The method of claim 24, wherein said detection reagent comprises an antibody.

32. The method of claim 24, wherein said detection reagent comprises said viral capsid of interest.

33. The method of claim 24, wherein said solid surface is selected from a group consisting of a microplate, resin, agarose beads, and magnetic beads.

34. The method of claim 24, wherein said solid surface is coated with avidin or streptavidin.

35. The method of claim 24, wherein said antibody against said viral capsid of interest is an anti-drug antibody.

36. The method of claim 24, wherein said viral capsid of interest is an AAV capsid.

37. The method of claim 36, wherein a serotype of said AAV capsid is AAV1, AAV2, AAV3, AAV4, AAV5, AAV6, AAV7, AAV8, AAVrh8, AAV2/8, AAV9, AAV10, AAV1, AAV12, a combination thereof, or a variant thereof.

38. The method of claim 36, wherein said AAV is a viral vector.

39. The method of claim 38, wherein said AAV is a therapeutic viral vector.

40. The method of claim 24, wherein said viral capsid is a nonenveloped viral capsid.

41. A composition for detecting anti-drug antibodies against an AAV vector, comprising:
(a) a sample;
(b) an AAV vector conjugated to biotin;
(c) an AAV vector conjugated to ruthenium; and
(d) a solid surface coated with avidin or streptavidin,
wherein said AAV vector conjugated to biotin and said AAV vector conjugated to ruthenium are modified forms of the same AAV vector.

42. The method of claim 41, wherein said sample is serum.

43. The method of claim 42, wherein said serum is human serum.

44. The method of claim 41, wherein said solid surface is selected from a group consisting of a microplate, resin, agarose beads, and magnetic beads.

45. The method of claim 41, wherein a serotype of said AAV vector is AAV1, AAV2, AAV3, AAV4, AAV5, AAV6, AAV7, AAV8, AAVrh8, AAV2/8, AAV9, AAV10, AAV1, AAV12, a combination thereof, or a variant thereof.

46. The method of claim 41, wherein said AAV vector is a therapeutic vector.

47. A method for producing a labeled viral vector, comprising:
(a) buffer exchanging a sample including a viral vector to produce a viral vector sample with basic pH;
(b) contacting said viral vector sample with basic pH to N-hydroxysuccinimide ester linked to a label to produce a mixed sample including labeled viral vector; and
(c) purifying said mixed sample to produce a labeled viral vector.

48. The method of claim 47, wherein said label is selected from a group consisting of a radiologic label, a photoluminescent label, a chemiluminescent label, a fluorescent label, a fluorophore, a hapten, an affinity label, an electrochemiluminescent label, and an enzyme label.

49. The method of claim 48, wherein said label is selected from a group consisting of biotin, ruthenium, and horseradish peroxidase.

50. The method of claim 47, wherein said viral vector is an AAV vector.

51. The method of claim 50, wherein a serotype of said AAV vector is AAV1, AAV2, AAV3, AAV4, AAV5, AAV6, AAV7, AAV8, AAVrh8, AAV2/8, AAV9, AAV10, AAV1, AAV12, a combination thereof, or a variant thereof.

52. The method of claim 47, wherein a concentration of said viral vector in said viral vector sample with basic pH is from about 10¹² to about 10¹³ vector genomes per mL.

53. The method of claim 47, wherein a molar ratio of said N-hydroxysuccinimide ester linked to a label to said viral vector is from about 50:1 to about 20,000:1.

54. The method of claim 53, wherein said molar ratio is about 10,000:1.

55. The method of claim 47, wherein said viral vector is a nonenveloped viral vector.

56. The method of claim 47, wherein said purification comprises buffer exchanging said mixed sample to remove unconjugated label.

57. A method for determining a degree of labeling of a viral capsid, comprising:
(a) determining a concentration of conjugated label in a labeled viral capsid sample;
(b) determining a concentration of viral capsid in said labeled viral capsid sample; and
(c) dividing the concentration of (a) by the concentration of (b) to determine a degree of labeling of said viral capsid.

58. The method of claim 57, wherein said viral capsid is an AAV capsid.

59. The method of claim 58, wherein a serotype of said AAV capsid is AAV1, AAV2, AAV3, AAV4, AAV5, AAV6, AAV7, AAV8, AAVrh8, AAV2/8, AAV9, AAV10, AAV1, AAV12, a combination thereof, or a variant thereof.

60. The method of claim 57, wherein said label is selected from a group consisting of a radiologic label, a photoluminescent label, a chemiluminescent label, a fluorescent label, a fluorophore, a happen, an affinity label, an electrochemiluminescent label, and an enzyme label.

61. The method of claim 60, wherein said label is ruthenium.

62. The method of claim 57, wherein determining a concentration of conjugated label comprises:
(a) subjecting said labeled viral capsid sample to size exclusion chromatography;
(b) measuring light absorbance at a wavelength correspond to an absorption maximum of said label;
(c) integrating the area under the curve corresponding to the main peak of the labeled viral capsid at the wavelength of (b) to determine a peak area; and
(d) comparing said peak area to a standard curve relating peak area to concentration to determine a concentration of said conjugated label.

63. The method of claim 62, wherein said wavelength is about 450 nm.
